(19) United States
(12) Patent Application Publication
(10) Pub. No.: US 2006/0142699 A1
(43) Pub. Date: Jun. 29, 2006

(54) Rotatable suture ring

(76) Inventor: Fred P. Lampropoulos, Sandy, UT (US)

Correspondence Address: WORKMAN NYDEGGER;(F/K/A WORKMAN NYDEGGER & SEELEY), 60 EAST SOUTH TEMPLE, 1000 EAGLE GATE TOWER, SALT LAKE CITY, UT, 84111, US

(21) Appl. No.: 11/021,513

(22) Filed: Dec. 23, 2004

(51) Int. Cl. A61M 5/178

(52) U.S. Cl. 604/164.04

(57) ABSTRACT

An introducer sheath having a rotation component allowing rotation of a suture ring. The rotation component having a first position allowing rotation of the suture ring and a second position which secures the rotational position of the suture ring. In one embodiment, the introducer sheath comprises a main body having a head portion and a base portion, wherein at least one of the head and base portions further comprise an attached suture ring. The suture ring can be secured to one or both of the head and base portions. In another embodiment, one or both of the head portion and the base portion can be rotated about an axis approximated by the sheath. In one embodiment, a detent prohibits the head portion and base portion from separating from one another.

### BACKGROUND OF THE INVENTION

1. The Field of the Invention

The present invention relates to medical introducer sheaths and related apparatuses. More particularly the present invention relates to a rotatable suture ring for use with an introducer sheath.

2. Background and Relevant Art

Present medical technologies include invasive and non-invasive devices for providing patients with localized medical treatment. One such device is called an introducer sheath, which is utilized to provide medical service practitioner access to the inside of a patient's body, such as through a patient's artery or vein. Once a portion of the introducer sheath is inserted into the patient, the introducer sheath allows bloodless access to the patient. In particular, guidewires, catheters, medicines and other articles or materials can be inserted into the patient through the introducer sheath.

A conventional introducer sheath includes a main body (or “housing”) that encloses an introducer valve, such as a hemostasis valve. A sheath is positioned at the distal end of the main body. Generally, a user operates the introducer sheath by puncturing the patient's skin, and then inserting the sheath of the introducer sheath into the patient's body (e.g., into a vessel). The sheath facilitates delivery of guide wires or other articles into the patient.

Some introducer sheaths also include a suture ring. The suture ring provides the introducer sheath with a securing point so that the introducer sheath can be affixed to the patient's body, or positioned securely nearby. For example, a user can insert a suture and needle through the suture ring and into a patient's skin, then thread the needle through the patient's skin and tie the suture ring securely.

One will appreciate that the position of the introducer sheath and suture ring on a given patient can have a significant impact on the ease by which a user can secure the introducer sheath proximate the patient. In particular, securing the suture ring as described is more easily managed when the suture ring is facing against the patient's skin, rather than facing away. Similarly, where the suture ring abuts the structure upon which the user intends to secure the suture ring securement of the suture ring to the structure is more easily accomplished.

A right-handed user may have the greatest ease accessing the introducer sheath: and suture ring when the suture ring is positioned on the patient at a desired angle or direction. Whereas, in another position, the right-handed user may have some difficulty accessing the introducer sheath, or may have difficulty reaching the suture ring to affix the introducer sheath on the patient. It will also be appreciated that convenience for a right-handed person in one position may create inconvenience for a left-handed user in the same position.

Some present approaches to provide a desired juxtaposition of introducer sheath elements have focused primarily on simply providing one left-handed and one right-handed version of the same introducer sheath to a user. This type of solution, however, can unnecessarily increase the cost of medical supplies when having to purchase both left and right-handed introducer sheaths. Additionally, a single apparatus may not be sufficiently adaptable to requirements of a procedure to be performed. Other present approaches to this problem have relied on allowing a segmented introducer sheath main body to rotate about an axis so that the introducer sheath can be configured both for right and left-handed users. Present rotating mechanisms, however, are difficult to adjust and/or are too easily rotated allowing rotation of the suture ring relative to introducer sheath body at inconvenient times. In particular, introducer sheaths that do include some type of rotation capability implement primarily a sliding rotation function that can fall out of alignment.

### BRIEF SUMMARY OF THE INVENTION

The present invention relates to an introducer sheath. More particularly, the present invention relates to systems and apparatus including a rotatable suture ring for use with an introducer sheath. In one embodiment, the suture ring includes a first position that allows the suture ring to be rotated and a second position that secures the rotational position of the suture ring relative to the introducer sheath. The suture ring allows a portion of the introducer sheath to which the suture ring is attached to be rotatable. This allows the suture ring to be more easily attached proximate a patient under a variety of use or space constraints.

The introducer sheath includes a sheath which is configured to be inserted, at least in part, into a patient's body. The sheath is connected to the main body of the introducer sheath. The introducer sheath can also include a side port which attaches to an entry line. In one embodiment, the main body is further segmented into a head portion and a base portion. A rotation mechanism on the main body interfaces between the head portion and the base portion. The rotation mechanism allows rotation of the head portion relative to the base portion. The head portion can also be selectively secured in a rotational position relative to the base portion to allow the user to minimize rotation of the head portion relative to the base portion. In one embodiment, the rotation mechanism can include any number of locking structures such as complementary ridges or grooves which allow securing of the rotatable position of the suture ring. Accordingly, an introducer sheath in accordance with embodiments of the present invention can be easily configurable for a variety of positions, such as positioning for both left and right-handed users, and can be adapted to various spatial constraints with minimal effort.

Additional features and advantages of the invention will be set forth in the description which follows, and in part will be obvious from the description, or may be learned by the practice of the invention. The features and advantages of the invention may be realized and obtained by means of the instruments and combinations particularly pointed out in the appended claims. These and other features of the present invention will become more fully apparent from the following description and appended claims, or may be learned by the practice of the invention as set forth hereinafter.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1A illustrates an introducer sheath in a fixed position.

FIG. 1B illustrates an introducer sheath in a rotated position.

FIG. 2A illustrates a cross-sectional view of a portion of a rotation mechanism utilized with an introducer sheath when the head portion and the base portion are engaged.

FIG. 2B illustrates the rotation mechanism of FIG. 2A when the head portion and the base portion are disengaged.

FIGS. 3A-3C illustrate depictions of a rotatable suture ring of an introducer sheath in which the introducer sheath is disengaged, rotated, and engaged.

FIGS. 4A and 4B illustrate detailed perspective views of rotation mechanisms in accordance with an alternative embodiment of the present invention.

### DETAILED DESCRIPTION OF THE PREFERRED EMBODIMENTS

The present invention relates to an introducer sheath. More particularly, the present invention relates to systems and apparatus including a rotatable suture ring for use with an introducer sheath. In one embodiment, the suture ring includes a first position that allows the suture ring to be rotated and a second position that secures the rotational position of the suture ring relative to the introducer sheath. The suture ring allows a portion of the introducer sheath to which the suture ring is attached to be rotatable. This allows the suture ring to be more easily attached proximate a patient under a variety of use or space constraints.

The introducer sheath includes a sheath which is configured to be inserted, at least in part, into a patient's body. The sheath is connected to the main body of the introducer sheath. The introducer sheath can also include a side port which attaches to an entry line. In one embodiment, the main body is further segmented into a head portion and a base portion. A rotation mechanism on the main body interfaces between the head portion and the base portion. The rotation mechanism allows rotation of the head portion relative to the base portion. The head portion can also be selectively secured in a rotational position relative to the base portion to allow the user to minimize rotation of the head portion relative to the base portion. In one embodiment, the rotation mechanism can include any number of locking structures such as complementary ridges or grooves which allow securing of the rotatable position of the suture ring. Accordingly, an introducer sheath in accordance with embodiments of the present invention can be easily configurable for a variety of positions, such as positioning for both left and right-handed users, and can be adapted to various spatial constraints with minimal effort.

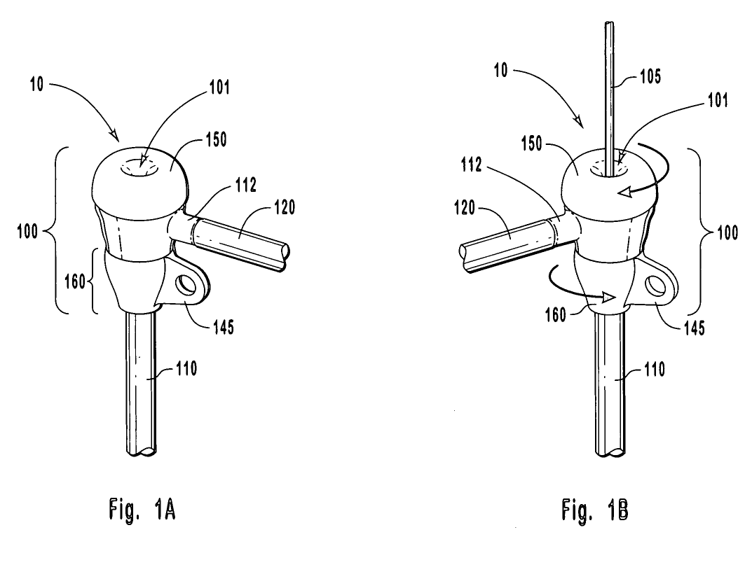

FIG. 1A illustrates an introducer sheath 10 having a main body 100 comprising a head portion 150 and a base portion 160. Introducer sheath 10 also includes a main bore 101, a sheath 110, and a side port 112. In the illustrated embodiment, main bore 101 allows a practitioner to thread guidewires, catheters, and other articles and materials into introducer sheath 10. Main body 100 houses an introducer valve such as a hemostasis valve that is positioned in a main lumen of introducer sheath 10. The introducer valve provides a fluid tight seal providing bloodless access to the patient through main bore 101 of introducer sheath 10. Typically the introducer valve is positioned in head portion 150 immediately adjacent main bore 101.

Sheath 110 is configured to be inserted, in whole or in part, into a patient. Typically sheath 110 is secured to base portion 160 of main body 100. Sheath 110 is in fluid connection with a main lumen positioned in main body 100. Sheath 110 can be inserted into an artery, vein, or other portion of the patient's body. Sheath 110 maintains open contact with the artery, vein, or other portion of the patient's body allowing the practitioner to insert and retract multiple guidewires, catheters, or other articles and materials, alone or in combination, without requiring multiple punctures of the patient.

Side port 112 is positioned on the side of main body 100. Side port 112 is in fluid communication with the main lumen of main body 100. Side port 112 allows introduction of blood, saline, medicines, and other articles and materials into the patient through the main lumen and sheath .110 of the introducer sheath 10. By being introduced through side port 112, introduction of such fluids, articles, and materials does not obstruct or interfere with aspects of the procedure performed through main bore 101. Delivery of fluids and materials through side port 112 occurs utilizing entry line 120. As will be appreciated by those skilled in the art, an introducer sheath 10 can be utilized without a side port 112 or entry line 120.

In general, an “entry line” will be understood to mean a flexible tubing (e.g., flexible surgical tubing), or a series of connected flexible tubing, that serves as a fluid delivery vehicle from a medical source to side port 112 of introducer sheath 10. In addition, a “medical source” (not shown) can be any container for housing medicinal or nutritional fluids such as, for example, an infusate bag containing saline solution, nutritional supplements, blood, plasma, medicines, and any other fluids or materials to be infused into a patient.

FIG. 1A also depicts a suture ring 145 as an attached appendage to base portion 160. In the illustrated embodiment, suture ring 145 allows a practitioner to secure the introducer sheath 10 at a desired position during a procedure. In one embodiment, suture ring 145 is attached to the patient using one or a plurality of sutures directly secured to the patient's skin or other body part. In another embodiment, suture ring 145 is secured with medical tape, adhesive, or other articles or material. Suture ring 145 can also be affixed to a-working surface or to another article or material in the surgical field.

Suture ring 145 is rotatable relative to one or more of the other components of the introducer sheath. In the illustrated embodiment, a rotatable mechanism is provided at the interface between head portion 150 and base portion 160. Because side port 112 is attached to head portion 150 and suture ring 145 is attached to base portion 160, the rotatable mechanism at the interface between head portion 150 and base portion 160 allows suture ring 145 to rotate relative to side port 112. This can allow the user to rotate either one or both of suture ring 145 and side port 112 relative to one another. This can be helpful when the positioning of side port 112 and entry line 120 interfere with securing of suture ring 145 to a patient. For example, in the embodiment illustrated in FIG. 1A, side port 112 and entry line 120 are positioned immediately above suture ring 145. In this position side port 112 can interfere with access to suture-ring 145 for securement by the practitioner. Additionally, the illustrated right handed: disposition of suture ring 145 may be difficult for a left handed practitioner to secure.

FIG. 1B illustrates rotation of suture ring 145 and/or side port 112 relative to FIG. 1A. A guide wire 105 is shown introduced through main bore 101. In the illustrated embodiment, the directional arrow proximate base portion 160 shows that suture ring 145 is rotating relative to head portion 150. Rotational movement of head portion 150 relative to base portion 160 allows repositioning of side port 112 relative to suture ring 145. In the illustrated position side port 112 is no longer positioned above suture ring 145. The repositioning of side port 112 relative to suture ring 145 provides easier access to suture ring 145 when the practitioner is securing suture ring 145 to the patient or other surface or material. Additionally, repositioning side port 112 can allow the user to manipulate the position of entry tubing 120 as desired during the procedure at being performed.

In the illustrated embodiment, the positioning of side port 112 in FIG. 1B has moved from the position depicted in FIG. 1A. In contrast, the positioning of suture ring 145 in FIG. 1B has not moved substantially from the positioning of suture ring 145 in FIG. 1A. As a result, suture ring 145 is configured to be secured in the right handed position. It will be appreciated that the positioning of side port 112 can be maintained on the right side of the introducer sheath while suture ring 145 is rotated to the left side of the introducer sheath. This can position suture ring 145 optimally for securement by a left handed practitioner. It will be appreciated that the positioning of suture ring 145 relative to main body 100 can be employed for reasons other than the dominant, hand of the practitioner performing the procedure.

A variety of types and configurations of suture ring and side port configurations can be utilized without departing from the scope and spirit of the present invention. For example, a suture ring that can rotate relative to both the head portion and the sheath can be utilized. In another embodiment, the suture ring rotates while the head portion and sheath of the introducer sheath remain fixed relative to one another. In another embodiment, the suture ring is affixed to a portion of the introducer sheath other than the base portion. In another embodiment, a rotating portion is provided between a head-portion and base portion and the suture ring is secured to the rotating portion.

One will appreciate that an introducer sheath can include multiple suture rings that are connected to other portions or segments of the introducer sheath main body, such as the head portion. Having multiple suture rings on the introducer sheath housing can be useful in the event, for example, that a user (e.g., a medical technician, nurse, physician, etc.) wishes to secure the given introducer sheath to a patient more securely, such as in a variety of angles, as well as securing the introducer sheath to the patient and another structural member (not shown).

As discussed above, FIG. 1B illustrates an overview perspective of the introducer sheath 10 shown in FIG. 1A, wherein head portion 150 and base portion 160 have been rotated with respect to each other. In particular, as will be discussed in greater detail in the following description, base portion 160 can be disengaged from a secured, or locking position, with respect to head portion 150, and then rotated with respect to head portion 150, and vice versa. Accordingly, a user can gain the space and use benefits of suture ring 145 without the disadvantages of slippage.

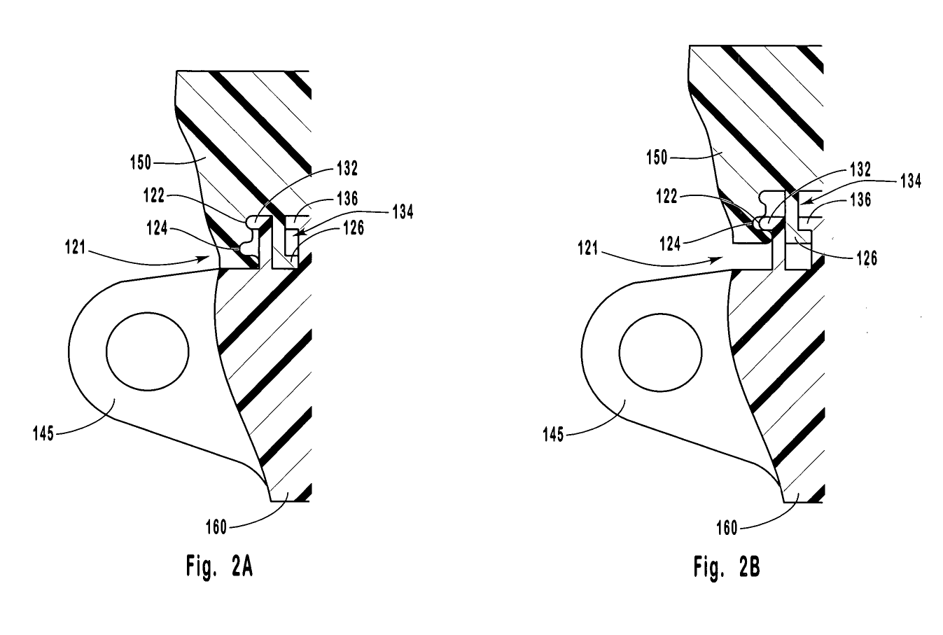

FIGS. 2A and 2B illustrate a rotation component 121 according to one embodiment of the present invention. In the illustrated embodiment, rotation component 121 includes differential cavities 122 and 124, securement ridge 126, locking flange 132, and rotational flange 136. Differential cavities 122, 124 and securement ridge 126 are positioned in head portion 150. Locking flange 132 and rotational flange 136 are positioned in base portion 160. Locking flange 132 is one example of a detent. Rotation component 121 is one example of a means for providing rotatable securement.

Differential cavities 122 and 124 are adapted to accommodate locking flange 132. Differential cavity 122 has a narrower cross section than differential cavity 124. The head of locking flange 132 is adapted to be selectively movable between differential cavity 122 and 124. When locking flange 132 is positioned in differential cavity 122, the head of locking flange 132 provides a secure coupling which controls rotational movement of head portion 150 relative to base portion 160. When locking flange 132 is positioned in differential cavity 124 a looser coupling is provided which allows rotational movement of the head of locking flange 132 relative to differential cavity 124. This also allows head portion 150 to rotate relative to base portion 160.

Rotational flange 136 is positioned in slot 134. Rotational flange 136 is configured to abut securement ridge 126 to maintain the coupling between head portion. 150 and base portion 160. The head of rotational flange 136 maintains a uniform contact with the wall of slot 134 when locking flange 132 is positioned in either differential cavity 122 or differential cavity 124. This helps maintain uniform and desired rotation of base portion 160 relative to head portion 150.

FIG. 2A illustrates a configuration in which head portion 150 and base portion 160 are engaged in a secure position. Locking flange 132 is positioned in differential cavity 122. Rotational flange 136 is positioned at the top of slot 134. The width of differential cavity 122 and the size of the head of locking flange 132 provide sufficient contact to minimize rotational movement of head portion 150 relative to base portion 160. In this configuration, locking flange 132 fits snugly within cavity 122, such that frictional forces prevent head portion 150 and base portion 160 from rotating with respect to each other at inopportune times during a procedure. In the illustrated embodiment, rotation component 121 is in a second position for securing the rotational position of suture ring 145.

FIG. 2B illustrates rotation component 121 when locking flange 132 has been moved from differential cavity 122 to differential cavity 124. The larger size of differential cavity 124 allows the head of locking flange 132 to move within differential cavity 124 permitting base portion 160 to rotate relative to head portion 150. Securement ridge 126 prevents passage of rotational flange 136 maintaining the coupling of head portion 150 relative to base portion 160. In other words, head portion 150 and base portion 160 can be disengaged relative to each other such that locking flange 132 fits loosely within larger cavity 124, as opposed to snugly within smaller cavity 122. Accordingly, head portion 150 and base portion 160 can rotate with respect to each other freely when disengaged, and suture ring 145 can be rotated into an appropriate position. The inner wall of securement ridge 126 abuts rotational flange 136, thus prohibiting complete separation of the components of introducer sheath 10. In the illustrated embodiment, rotation component 121 is in a first position for allowing rotational movement of suture ring 145 relative to the other components of the introducer sheath.

The user can selectively move locking flange 132 from differential cavity 122 and 124 by simply grasping head portion 150 and base portion 160 and pulling the head portion 150 and base portion 160 in opposite directions. The user can move locking flange 132 from differential cavity 124 to differential cavity 122 by pushing head portion 150 and base portion 160 toward each other.

The configuration of rotation component 121 allows the user to change the rotational position of suture ring 145 relative to head portion 150. To rotate head portion 150 relative to base portion 160, the user simply grasps head portion 150 and base portion 160 pulling head portion 150 in the opposite direction of base portion 160. This moves locking flange 132 from differential cavity 122 to differential cavity 124. The user can then freely rotate head portion 150 relative to base portion 160. Once suture ring 145 is in a desired position around the circumference of the introducer sheath, the user pushes head portion 150 in the direction of base portion 160. This moves the head of locking flange 132 from differential cavity 124 to differential cavity 122 effectively securing the rotational position of suture ring 145.

In the illustrated embodiment, differential cavities 122, 124, securement ridge 126, locking flange 132, slot 134, and rotational flange 136 are features of circular flanges which encompass the entire diameter of the head portion 150 and base portion 160. This provides unlimited rotation ability (i.e. 360° degrees), although varying lesser amounts of rotation (i.e. 10-360 degrees) may be provided, depending on a manufacturer's preferences.

As will be appreciated by those skilled in the art, a variety of different types and configurations of rotation mechanisms can be utilized without departing from the scope and spirit of the present invention. For example, in one embodiment, the rotational flange and the locking flange are continuous for the entire circumference of the introducer sheath. In another embodiment, the locking flange and/or wall between the upper and lower differential cavities are slightly deformable to allow for movement of the locking flange between the differential cavities. Locking flange 136 is one example of a detent.

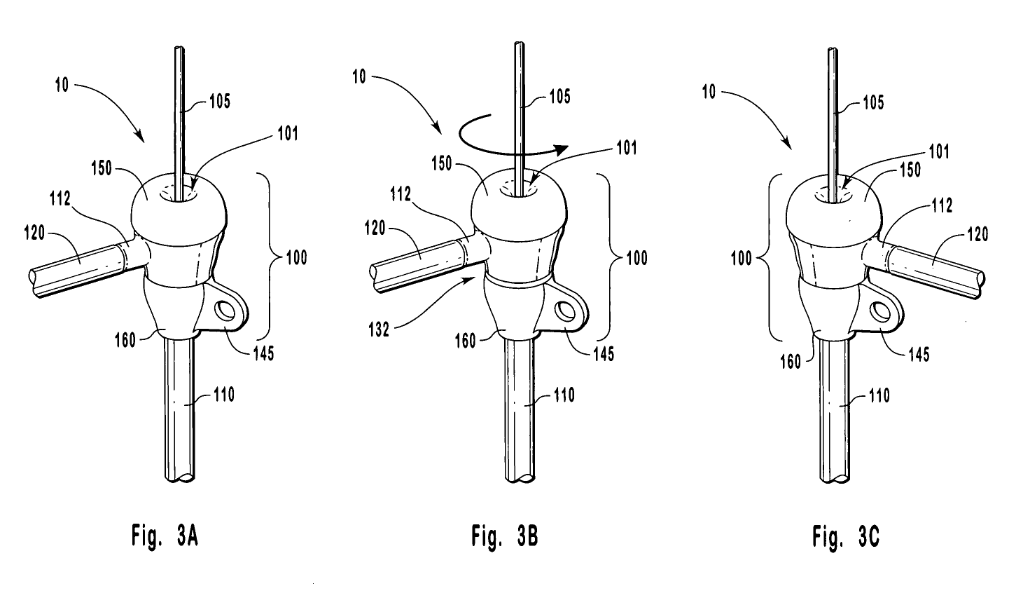

FIGS. 3A-3C illustrate an exemplary sequence for rotating head portion 150 and base portion 160 of the introducer sheath 10. In particular, FIG. 3A shows an engaged position of an introducer sheath 10 such that side port 112 and the suture ring 145 are out of alignment. By contrast, FIG. 3B illustrates that the head portion 150 and base portion 160 can be disengaged with respect to the other, such as by lifting head portion 150, or by lowering the base portion 160. As discussed with reference to FIGS. 2A and 2B, this moves locking flange 132 from differential cavity 122 to differential cavity 124 (see FIGS. 2A and 2B). Head portion 150 and/or base portion 160 can then rotate in either a clockwise or counterclockwise direction about an axis that approximates the circumference of sheath 110. In the illustrated embodiment, when head portion 150 and base portion 160 are separated, a user can see an inner wall of locking flange 132 from an outside perspective.

After the user has rotated head portion 150 (see FIG. 3C) with respect to base portion 160 or vice versa, the user can then engage head portion 150 and base portion 160 in a secure fashion. For example, a user can then compress head portion 150 and base portion 160 such that locking flange 132 fits snugly within the smaller differential cavity 122 (see FIG. 2A). Accordingly, suture ring 145 is secured in a different orientation with respect to side port 112 than shown in FIG. 3A. The reoriented introducer sheath 10 can then be used with the patient as desired.

In short, one will appreciate that there can be a variety of embodiments for providing rotation of suture ring 145. In the illustrated embodiment, head portion 150 and base portion 160 are rotatable relative to one another. In another embodiment, the suture ring is rotatable independently of the head and base portion. Furthermore, there can be a variety of securement mechanisms that prohibit slippage, or any other type of accidental rotation between the head portion and base portion after both portions have been engaged, or secured.

The rotational mechanism can be sufficiently secure once the head portion 150 and base portion 160 are positioned together, to prevent the head portion 150 and base portion 160 from slipping out of alignment. For example, any number of friction-based mechanisms between abutting surfaces of the head portion 150 and base portion 160 can secure the respective portions from rotation. In addition, the introducer sheath 10 can also be configured to implement alternative securing mechanisms to prevent rotation when engaged, such as corresponding tooth and groove formations.

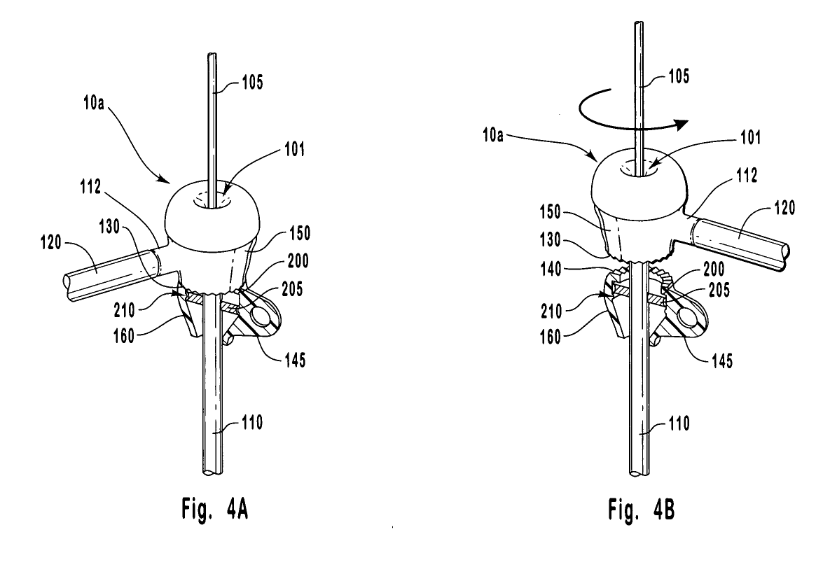

For example, FIG. 4A illustrates an alternative embodiment for securing an introducer sheath 10a when engaged, and also alternative mechanisms for rotating a given introducer sheath when disengaged. In particular, an introducer sheath 10a can include radial groove and detent means. In particular, base portion 160 can comprise a radial, inwardly extending rib 210 formed against an internal wall. A grooved member 205 can extend radially outward from sheath 110. In the illustrated embodiment, rib 210 comprises a part of base portion 160 and grooved member 205 comprises a part of head portion 150.

When head portion 150 is engaged with base portion 160, grooved member 205 will be positioned around rib 210, such that rib 210 will hold the grooved member 205 in place. Nevertheless, rib 210 is flexible or small enough such as to not prohibit disengagement of grooved member 205 from rib 210. As such, a manufacturer may desire to form rib 210 using a primarily flexible material such as flexible plastic or rubber materials. As such, rib 210 can be configured to provide a secure but flexible detent mechanism.

Additionally, base portion 160 can further comprise an inflexible flange 200, which can extend further radially inward toward sheath 110. Grooved member 205 abuts flange 200 when head portion 150 and base portion 160 are disengaged. Accordingly, flange 200 provides a mechanism for prohibiting head portion 150 and base portion 160 from complete separation. Thus a manufacturer may desire to form rib 210 using a primarily rigid material such as rigid plastic or metallic materials.

As shown in FIGS. 4A and 4B, introducer sheath 10a includes additional mating securement components 130 and 140 formed at opposing surfaces of the head portion 150 and the base portion 160, thus helping to further secure the head portion 150 and base portion 160 from slippage when engaged. For example, a lower surface of head portion 150 can comprise alternating tooth and groove formations extending downward, which fit into corresponding tooth and groove formations on an upper surface of the base portion 160. Such interlocking tooth and groove configurations, however, are exemplary only. Accordingly, one will appreciate that there can be numerous configurations for interlocking the head portion 150 and base portion 160 when engaged.

Continuing with FIG. 4B, suture ring 145 of introducer sheath 10a can be rotated with respect to head portion 150 by disengaging head portion 150 from base portion 160 such that the grooves of grooved member 205 slip past the rib 210. Head portion 150 and/or base portion 160 can then be rotated about the axis approximated by sheath 110, as appropriate. When the user has rotated head portion 105 and/or base portion 160 so that suture ring 145 is in a desired position, the user can then compress head portion 150 and base portion 160 together such that the reciprocal securement members 130 and 140 interlock. Furthermore, grooved member 205 will be pressed back in place such that it is secured by rib 210. As such, suture ring 145 is now in a rotated and secured position relative to head portion 150.

Accordingly, the described rotation capability of the introducer sheath, in particular with respect to the suture ring, allows a high degree of configurability and positioning with the present introducer sheath. For example, at least this advantage allows the introducer sheath to be particularly suited to both left and right-handed users with minimal interference to a patient. In addition, it will be understood that the introducer sheath can be easily adapted to include multiple entry lines, multiple suture. F-rings, and multiple rotator mechanisms adjoining multiple other segments of the introducer sheath. The present invention therefore provides an introducer sheath having a high degree of configurability, particularly with respect to positioning a given introducer sheath on a patient.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

I claim:

1. An introducer sheath for providing bloodless access to the vasculature of a patient, the introducer sheath comprising:
a main body having a first side and a second side and defining an interior chamber interposed between the first and second sides;
a suture ring positioned adjacent at least one of the first side or the second side of the main body, the suture ring configured to secure the introducer sheath to the patient or other object within the medical field;
a rotation component utilized in connection with the suture ring, wherein the rotation component has a first position configured to allow the suture ring to rotate relative to the main body and a second position configured to secure the rotational position of the suture ring around the circumference of the main body of the introducer sheath.

2. The introducer sheath of claim 1, wherein the main body includes a head portion and a base portion.

3. The introducer sheath of claim 2, wherein the suture ring is affixed to at least one of the base portion or the head portion.

4. The introducer sheath of claim 3, wherein the rotation mechanism allows the base portion to rotate relative to the head portion.

5. The introducer sheath of claim 1, wherein the rotation mechanism includes differential cavities and a locking flange.

6. The introducer sheath of claim 5, wherein a head of the locking flange moves between a first differential cavity and a second differential cavity when the rotation mechanism moves from the first position to the second position.

7. The introducer sheath of claim 6, wherein the first differential cavity has a smaller cross-section than at the second differential cavity.

8. The introducer sheath of claim 7, wherein the head of the locking flange is positioned in the first differential cavity having a smaller cross-section when in the secured position.

9. The introducer sheath of claim 7, wherein the head of the locking flange is positioned in the second differential cavity having the large cross-section when in the rotational position.

10. An introducer sheath comprising:
a head portion having at least a first cavity and a second cavity formed within an inner wall such that the first cavity is smaller cross-section than the second cavity;
a base portion having at least one locking flange formed within an inner base wall such that the at least one locking flange fits tightly within the first cavity when the head portion and the base portion are engaged, such that the at least one locking flange fits loosely within the second cavity when the head and base portion are disengaged; and
a suture ring attached to one of the head portion and the base portion.

11. The introducer sheath as recited in claim 1, wherein the introducer sheath includes a plurality of suture rings attached thereto.

12. The introducer sheath as recited in claim 10, wherein the introducer sheath includes one or more side ports.

13. The introducer sheath as recited in claim 10, further comprising a sheath positioned at a distal end of the introducer sheath, the sheath approximating an axis around which any of the head portion and the base portion can rotate.

14. The introducer sheath as recited in claim 10, further comprising a securement ridge extending from a first inner wall of the head portion, wherein the securement ridge abuts an outwardly extending detent formed on an inner wall within the base portion when the head portion and base portion are disengaged, such that the head portion and the base portion cannot be completely separated.

15. The introducer sheath as recited in claim 10, further comprising one or more securement members extending from the head portion toward the base portion, and one or more reciprocal securement members extending from the base portion toward the head portion.

16. The introducer sheath as recited in claim 15, wherein the securement members comprise one or more detents extending from the head portion, and the reciprocal securement members comprise two or more corresponding recesses formed within the base portion.

17. An introducer sheath comprising:
a segmented main body having a head portion and a base portion positioned along a rotational axis;
a suture ring coupled to at least one of the head portion or the base portion; and
means for providing rotatable securement, the means for providing rotatable securement disposed between the head and base portions, such that the head and base portions can rotate relative to one another about the rotational axis when the head and base portions are disengaged, and such that the means for providing rotatable securement prohibits the head and base portions from rotating relative to one another about the rotational axis when the head and base portions are engaged.

18. The introducer sheath as recited in claim 17, wherein the means for providing rotatable securement comprises:
at least first and second cavities formed within an inner wall of the head portion such that the first cavity is shallower than the second cavity;
at least one detent formed within a first inner wall of the base portion such that the at least one detent fits tightly within the first cavity when the head portion and the base portion are engaged, and such that the at least one detent fits loosely within the second cavity when the head and base portion are disengaged.

19. The introducer sheath as recited in claim 17, further comprising an internal, releasable detent configured such that the first portion can be disengaged from the second portion sufficient to allow rotation of any of the first portion and second portion, while prohibiting the first portion from being completely separated from the second portion.

20. The introducer sheath as recited in claim 19, wherein the internal, releasable detent comprises:
a rib extending from a second inner wall of the head portion; and
a detent extending from a second inner wall of the base portion, the detent abutting the rib when the head portion and base portion are disengaged, such that the head portion and the base portion cannot be completely separated.

21. The introducer sheath as recited in claim 20, wherein the rib extends radially inward from the second inner wall of the head portion, and wherein the detent extends radially outward from the second wall of the base portion.
